(19) United States
(12) Patent Application Publication
(10) Pub. No.: US 2021/0085993 A1
(43) Pub. Date: Mar. 25, 2021

(54) ORAL HYGIENE SYSTEM AND METHOD OF USE

(71) Applicant: PhotoDynamic Inc., Halifax, NS (US)

(72) Inventors: Mark Tullio Morreale, Toronto (CA); Martin Labrecque, Toronto (CA); Sherri McFarland, Haslet, TX (US); Colin G. Cameron, Haslet, TX (US); Martin Greenwood, Dartmouth (CA); S. Alexander McLellan, Ketch Harbour (CA); Christopher David Gillespie, Toronto (CA)

(21) Appl. No.: 17/026,659

(22) Filed: Sep. 21, 2020

Related U.S. Application Data
(60) Provisional application No. 62/903,136, filed on Sep. 20, 2019.

(51) Int. Cl. A61N 5/06; A61N 5/10; A61N 5/02; A61C 17/22

(52) U.S. Cl. CPC A61N 5/0603; H02J 7/02; A61N 5/1014; A61N 5/022; A61C 17/228; A61N 2005/0606; A61N 2005/0666; A61N 2005/0651; A61N 2005/0643; A61N 2005/0659; A61N 2005/0661; A61N 2005/0662; A61N 2005/0626; A61N 2005/0628; A61C 2204/002; A61N 5/0624

(57) ABSTRACT

An oral hygiene device comprises a housing having an outside mouth portion and an inside mouth portion, at least one energy delivery element disposed in or on the inside mouth portion, configured to deliver energy to one or more teeth of a subject, a controller disposed in the outside mouth portion electrically connected to the at least one energy delivery element, an energy storage unit disposed in the outside mouth portion, and a sleeve removably connected to the inside mouth portion of the housing, the sleeve having upper and lower channels configured to accept the upper and lower teeth of the subject. A method of using an oral hygiene device and a method of administering an oral hygiene treatment are also described.

### CROSS-REFERENCE TO RELATED APPLICATIONS

This application claims priority to U.S. Provisional Patent Application No. 62/903,136, filed on Sep. 20, 2019, incorporated herein by reference in its entirety.

### BACKGROUND OF THE INVENTION

Treatments such as teeth cleaning, plaque removal and treatment of bacteria (e.g. bacterial infections, bad breath) often require a subject to visit a dental professional to manually apply a treatment. Dental procedures require a subject to remain open-mouthed while the practitioner performs a therapy. Such in-person cleanings are cumbersome, painful, can cause anxiety, and are typically expensive for a subject in terms of cost and time.

Home treatments for teeth cleaning are mostly aimed at whitening teeth, and therefore typically use chemical agents, sometimes in combination with light or other stimulation. Such treatments are often less effective than professional procedures, using for example a lower concentration of the cleaning or bleaching agents in order to reduce the risk of harm to the subject from accidental misuse. Consequently, such therapies often take longer to achieve the same result as a professional treatment. In addition, because existing teeth cleaning devices and methods are primarily concerned with whitening, the existing devices and methods may focus exclusively on the front teeth visible when the subject is smiling and may not clean the back teeth as effectively or at all.

It is well known that cleaning around braces is difficult and oral hygiene instructions are poorly followed by teens, who comprise the majority of those wearing braces. Poor oral hygiene can lead to longer orthodontic treatment due to the need to pause and remove wires, refer for extra cleanings, and may result in poor aesthetic outcomes due to enamel demineralization, and may negatively affect gum health, etc.

Where light therapies are used, power may be applied to light devices indiscriminately, for example allowing the light devices to emit light when not properly positioned in the subject's mouth. Such accidental activation of the lights, which may often be quite bright, can lead to discomfort and also a waste of power.

Enforcing or monitoring a subject's compliance with an in-home oral therapy routine may also be difficult, because existing devices have no way of tracking or recording when they are used and for how long. Dental professionals who wish to monitor a subject's compliance with a treatment regimen may be left with no alternative beyond asking the subject to self-report when and for how long they used a device.

Finally, existing in-home oral therapy devices are complex and require frequent cleaning and sanitization after use. For example, the device of U.S. patent application Ser. No. 10/944,740, filed Sep. 21, 2004 and incorporated herein by reference is constructed as a single piece with a power source connected to a mouthpiece having lighting elements for whitening teeth. The device of U.S. patent application Ser. No. 11/579,916, filed Mar. 16, 2005 and incorporated herein by reference is another example of a device constructed as a single piece having electronics and light sources for positioning in a subject's mouth. Cleaning such devices may be difficult, because water may accidentally enter the inside of the housing and interfere with the electronics. Furthermore, because mouths vary in size, a single-piece device must either be wholly custom made for a subject, or suffer from non-optimal fit.

Therefore, there is a need in the art for an inexpensive, effective in-home oral therapy device capable of use by those wearing braces, and having a compliance monitoring system, that is safe, efficient, and able to be customized for an ideal fit across multiple subjects. The present invention satisfies this need.

### SUMMARY OF THE INVENTION

In one aspect, an oral hygiene device comprises a housing having an outside mouth portion and an inside mouth portion, at least one energy delivery element disposed in or on the inside mouth portion, configured to deliver energy to one or more teeth of a subject, a controller disposed in the outside mouth portion electrically connected to the at least one energy delivery element, an energy storage unit disposed in the outside mouth portion, and a sleeve removably connected to the inside mouth portion of the housing, the sleeve having upper and lower channels configured to accept the upper and lower teeth of the subject.

In one embodiment, the device further comprises at least one control element disposed on the outside mouth portion. In one embodiment, the device further comprises a wireless communication interface electrically connected to the controller and configured to facilitate communication between the controller and computing device external to the oral hygiene device. In one embodiment, the device further comprises at least one indicator element disposed in the outside mouth portion. In one embodiment, the indicator element is an LED. In one embodiment, the at least one energy delivery element is a plurality of LEDs. In one embodiment, the LEDs are configured to emit light in a range of 200 nm to 1500 nm. In one embodiment, the LEDs are configured to emit light in a range of 400 nm to 500 nm. In one embodiment, the LEDs are configured to deliver a light intensity of at least 30 mW/cm² to teeth of the subject. In one embodiment, the device further comprises a temperature sensor disposed in the inside mouth portion and communicatively connected to the controller, wherein the controller is configured to interrupt a treatment if a temperature measured by the temperature sensor exceeds a threshold. In one embodiment, the device further comprises at least one environmental sensor disposed in the inside mouth portion and communicatively connected to the controller, wherein the controller is configured to start a treatment if at least one measurement obtained from the at least one environmental sensor indicates that the oral hygiene device is positioned in a mouth of the subject. In one embodiment, the at least one environmental sensor comprises a light sensor. In one embodiment, the energy storage unit is a rechargeable battery. In one embodiment, the device further comprises a wireless charging system configured to charge the battery. In one embodiment, the sleeve includes at least one reflective element. In one embodiment, the at least one energy delivery element is positioned in the occlusal plane of the teeth of the subject. In one embodiment, the at least one energy delivery element comprises a first energy delivery element configured to deliver energy to a facial surface of the teeth of the subject, and a second energy delivery element configured to deliver energy to a lingual surface of the teeth of the subject. In one embodiment, the at least one energy delivery element is configured to deliver pulsed energy to the one or more teeth of the subject. In one embodiment, the energy delivery element is configured to deliver electromagnetic energy in a spectrum selected from the group consisting of gamma rays, x-rays, ultraviolet light, visible light, infrared light, microwaves, and radio waves.

In another aspect, a method of using an oral hygiene device comprises applying a light activated treatment material to an oral hygiene device, waiting for an indication that the oral hygiene device is ready to begin treatment, activating a control element on the oral hygiene device to start an oral hygiene treatment, inserting the oral hygiene device into a mouth of a subject, waiting for an indication that the treatment is complete, and removing the oral hygiene device from the mouth.

In one embodiment, the method further comprises the step of applying a sleeve to the oral hygiene device. In one embodiment, the method further comprises the step of obtaining a sleeve custom manufactured for the subject or fitted to the subject from a set of standard sized sleeves. In one embodiment, the method further comprises the step of charging an energy storage unit in the oral hygiene device.

In another aspect, a method of administering an oral hygiene treatment comprises waiting for a signal indicating that a device has been removed from a charger, indicating to a subject when the device is ready to begin treatment accepting a user input from the subject indicating the beginning of treatment, supplying electrical power to one or more energy delivery elements at a first power level, waiting for one or more signals which, individually or in combination, indicate that the device is positioned in the subject's mouth, supplying electrical power to the one or more energy delivery elements at a second power level, waiting for a predetermined time period, turning off the one or more energy delivery elements, and indicating to the subject via an indicating means that the treatment is complete.

In one embodiment, the predetermined time period is two minutes or less. In one embodiment, the method further comprises monitoring a temperature measured by a temperature sensor positioned on the device and turning off the one or more energy delivery elements when the temperature exceeds a threshold.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is an oral hygiene device;

FIG. 1B is an oral hygiene device and a detached removable sleeve;

FIG. 1C is a perspective view of an exemplary oral hygiene device;

FIG. 1D is a front view of an exemplary oral hygiene device;

FIG. 1E is a back view of an exemplary oral hygiene device;

FIG. 1F is a right side view of an exemplary oral hygiene device;

FIG. 1G is a left side view of an exemplary oral hygiene device;

FIG. 1H is a top view of an exemplary oral hygiene device;

FIG. 1J is a bottom view of an exemplary oral hygiene device;

FIG. 2 is an oral hygiene device;

FIG. 3A is a detail view of an oral hygiene device;

FIG. 3B is a view of an attachment mechanism for an oral hygiene device;

FIG. 3C is a view of a removable sleeve of an oral hygiene device having a reflective element.

FIG. 3D is a cross-sectional view of a removable sleeve of an oral hygiene device.

FIG. 4 is an exploded view of an oral hygiene device;

FIG. 5A is an exemplary oral hygiene device;

FIG. 5B is an exemplary oral hygiene device with the replaceable sleeve removed;

FIG. 5C is a diagram of viewing angles of illumination devices positioned on an oral hygiene device.

FIG. 6A is an exemplary oral hygiene device;

FIG. 6B is an exemplary oral hygiene device with the replaceable sleeve removed;

FIG. 6C is an overhead view of an exemplary oral hygiene device;

FIG. 6D is a cross-sectional view of a removable sleeve of an exemplary oral hygiene device;

FIG. 7 is a system diagram of an oral hygiene device;

FIG. 8 is a method of the invention;

FIG. 9 is an exemplary software diagram of an oral hygiene device; and

FIG. 10 is an exemplary state machine diagram of an oral hygiene device.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

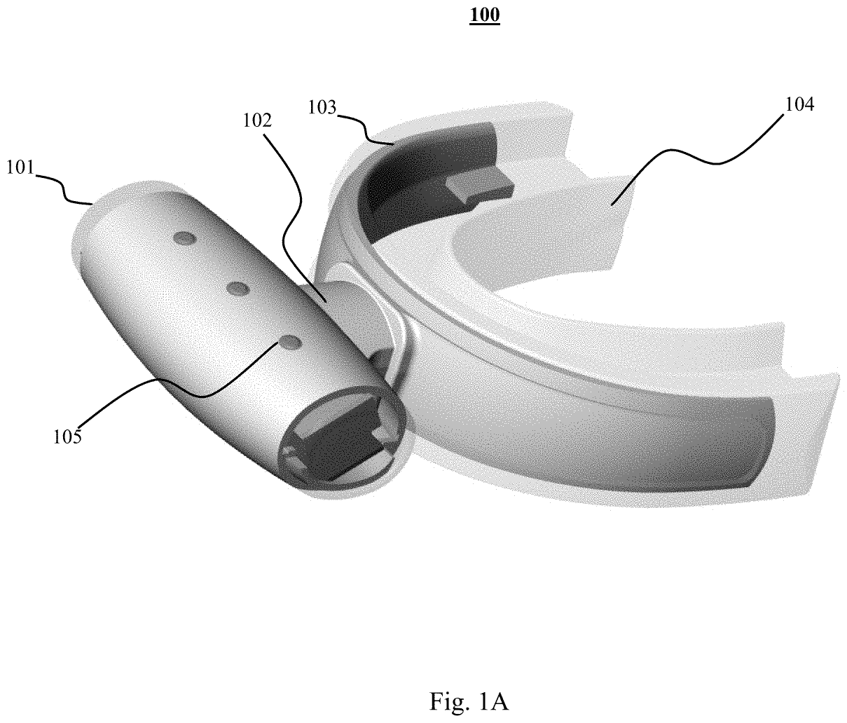

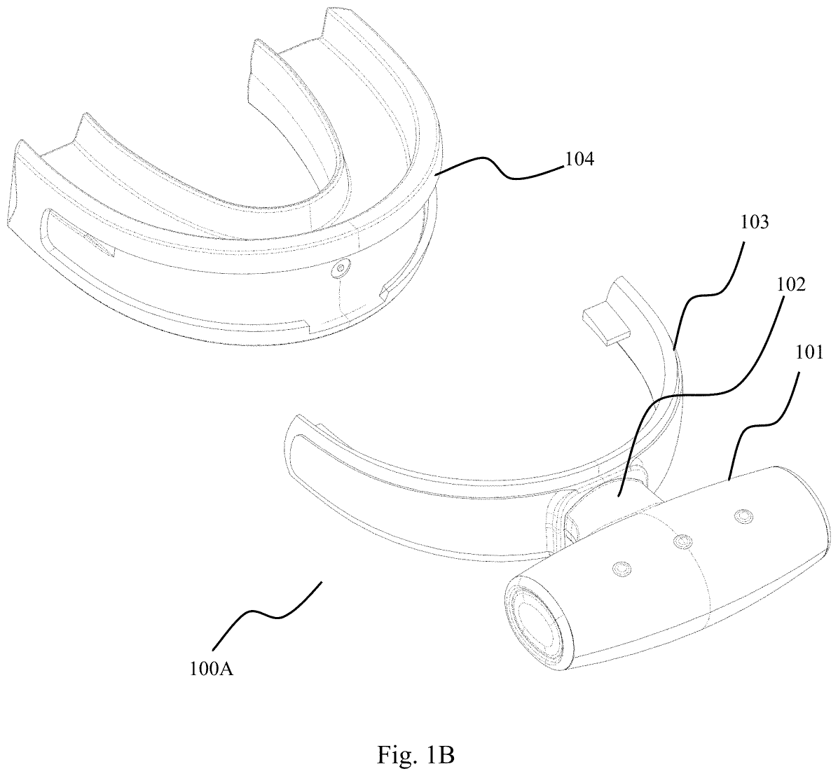

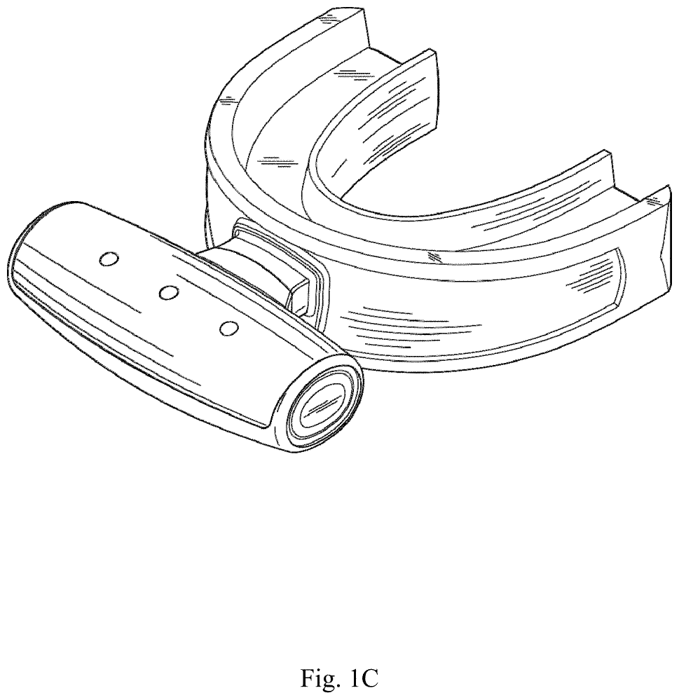

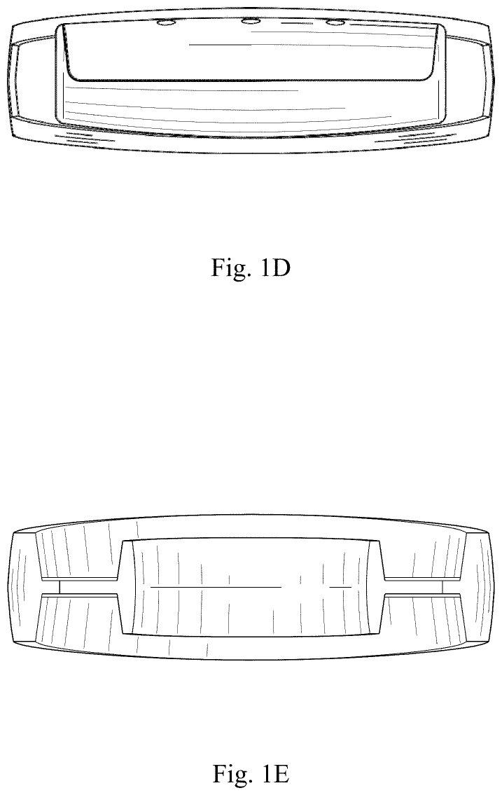

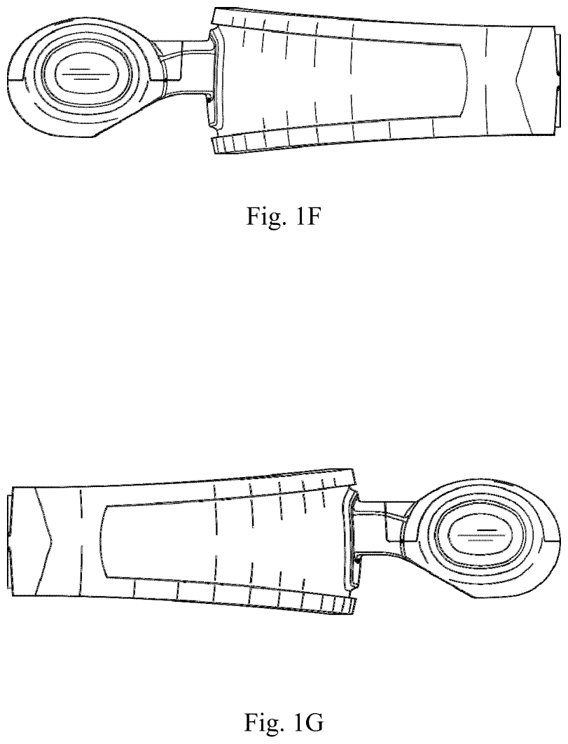

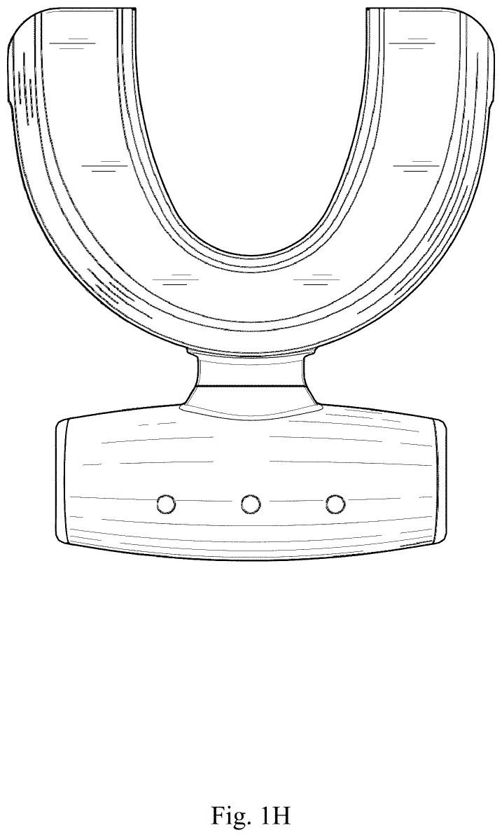

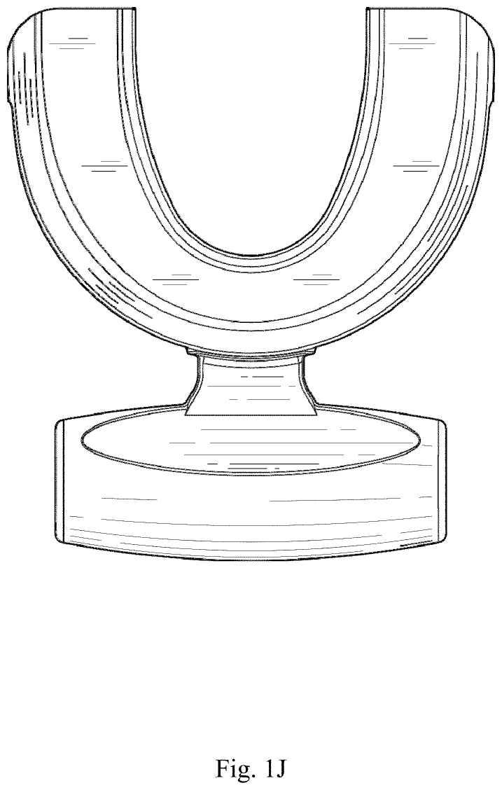

Aspects of the present invention relate to a light-activated antimicrobial system for oral hygiene. An exemplary device of the present invention is shown in FIG. 1A. With reference now to FIG. 1A, a device 100 includes handle 101 which is directly connected to neck 102 and illumination strip 103. In some embodiments, handle 101, neck 102, and illumination strip 103 are contained within the same housing, but in other embodiments may be contained in multiple housings that may be detachably connected to one another, for example with a snapping mechanism, one or more fasteners, magnets, or adhesive. One or more or all of the housings may be constructed from any suitable material, for example a plastic, including but not limited to polyolefin, polystyrene, or nylon, or a cast material, for example urethane. Also shown in FIG. 1A is disposable/replaceable sleeve 104, which is removably connected to illumination strip 103. Sleeve 104 is configured to receive a subject's teeth, wherein a subject bites down on the sleeve. During use, some or all of the illumination portion may be positioned in the subject's mouth, with the neck 102 and handle 101 protruding from the subject's mouth. Sleeve 104 may comprise multiple materials, for example a strip of light activated treatment material or photoactive anti-microbial foam disposed on the surface(s) of sleeve 104 making contact with the subject's teeth. In some embodiments, sleeve 104 is custom shaped for a subject's mouth, or may be selected from a set of standard sized sleeves in order to fit the subject. In some embodiments, sleeve 104 is produced via additive manufacturing or molding.

Handle 101 may include one or more indicator or control elements 105. Elements 105 may comprise LEDs, for example indicator LEDs, and/or may comprise buttons, switches, dials, digital displays, vibration buzzers, speakers, buzzers, or any other suitable components electrically connected to a controller and configured to indicate a status of the device or accept user input to control the device. Exemplary status indications include, but are not limited to, treatment about to begin, treatment completed, lighting system status or lighting system failure, charging status, charging complete, low battery, device in use, device not ready, treatment progress or stage, Bluetooth pairing, Bluetooth pair successful, reminders to initiate treatment, general error, etc.

Exemplary control signals may include, but are not limited to, initiating Bluetooth pairing, starting treatment, turning the device on and off, checking battery charge level, adjusting light intensity, and initiating data transfer.

With reference now to FIG. 1B, another view of an exemplary oral hygiene device is shown. The view of FIG. 1B shows the separation between the illuminator unit 100A, (which comprises the handle 101, the neck 102, and the illumination strip 103), and the sleeve 104, which may be removed from the illuminator unit 100A. The illumination strip 103 may comprise one or more light emitting elements, for example LEDs. Various embodiments of an illumination strip may comprise between 1 and 200 individual LED elements, or between 1 and 100 individual LED elements. In some embodiments, an illumination strip may comprise between 20 and 40 LED elements. In some embodiments, an illumination strip may comprise one or more flexible illumination elements, for example flexible OLED elements configured to illuminate one or more teeth of a subject. In some embodiments, an illumination strip may comprise fewer LED elements, with the light being distributed across the teeth of a subject via one or more light pipes or other refractive or reflective elements positioned within the sleeve 104 or within illumination strip 103. In some embodiments, the sleeve 104 may additionally contain one or more light emitting elements, and the interface between the sleeve 104 and the illumination strip 103 may comprise an electrical connection for providing power to the LEDs in the sleeve 104.

Illumination strip 103 may further comprise a transparent cover or lens positioned over the LED elements, between the one or more LED elements and the sleeve 104. In some embodiments, the transparent cover or lens is placed in direct contact with the sleeve 104 when the sleeve 104 is connected to illumination strip 103. The cover may be overmolded or may be a separate piece removably or fixedly attached to the illumination strip 103. In some embodiments, the transparent cover or lens may include features or textures configured to distribute or diffuse light from the LED elements.

Sleeve 104 may be made from any suitable material, but in some embodiments comprises silicone, thermoplastic, polyolefin elastomers (POE), polyurethane, styrene-ethylene-butylene-styrene (SEBS), styrene-ethylene-pro9pylene-styrene (SEPS), Liquid Silicone Rubber (LSR) or a combination of these. In some embodiments, the sleeve may comprise Ethyl-Vinyl Acetate (EVA) or a light cured resin. In some embodiments, the sleeve may be transparent or substantially transparent to a range of wavelengths of light, for example blue light. In some embodiments, a sleeve may be manufactured from a material that is transparent to light in a range of 200-1500 nm, 300-780 nm, 400-500 nm, 425-475 nm, or 440-460 nm. In some embodiments, a sleeve may be manufactured from a material that is transparent to all visible light. In some embodiments, a sleeve may be manufactured from a material that is transparent to all visible light and/or infrared or ultraviolet light. In some embodiments, a sleeve may be manufactured from a material that is translucent to all visible light and/or infrared or ultraviolet light.

In some embodiments, sleeve 104 may comprise a light activated treatment material, for example an antibacterial or antimicrobial foam, including but not limited to Japanese Knotweed (polygonum cuspidatum). The material may comprise a natural or synthetic photosensitizer or photo-active natural extract, which when exposed to light produces an immediate antimicrobial action. In other embodiments, a foam may be added to the sleeve 104, for example applied to sleeve 104 before a treatment. Sleeve 104 may be manufactured from a material that resists discoloration caused by contact with the foam. The foam may be configured to reduce plaque formation in patients wearing braces on the facial surface of their teeth, and/or may advantageously reduce gingivitis, puffy gums, prevent caries, or prevent demineralization of enamel.

In some embodiments, sleeve 104 may have a durometer hardness of between Shore A 20 and Shore A 70, or between Shore A 40 and Shore A 60. In some embodiments, sleeve 104 may be manufactured such that it maintains its shape after repeated application of force by the teeth of a subject. In some embodiments, sleeve 104 may comprise a deformable material that deforms and maintains the deformed shape after a fixed force is applied with the teeth of a subject. In some embodiments, the sleeve may be configured for use with dentures or orthodontic appliances.

With reference now to FIGS. 1C-1J, various views of an exemplary oral hygiene device are shown.

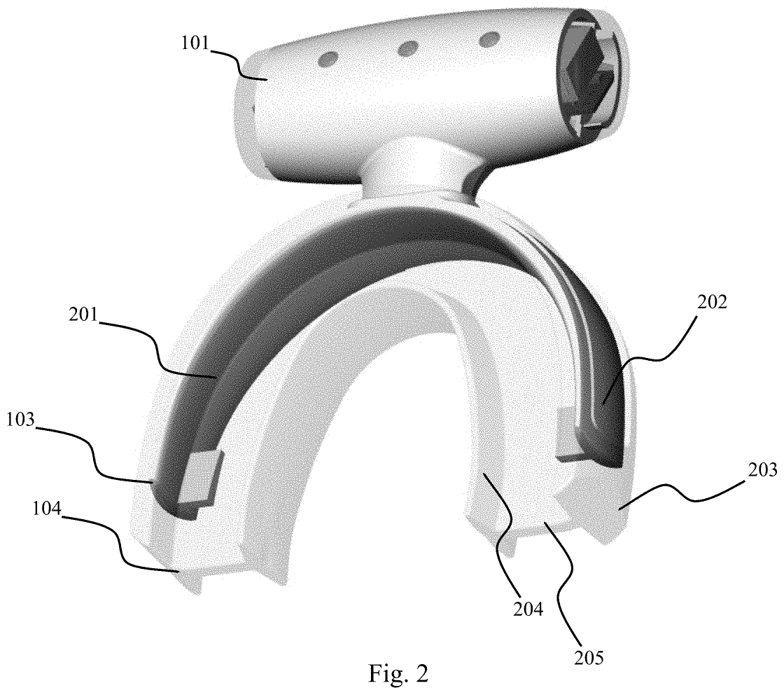

With reference now to FIG. 2, another view of an oral hygiene device is shown. FIG. 2 shows in detail the illumination strip having outer 202 and inner 201 surfaces, with the inner surface 201 in contact with sleeve 104. In some embodiments, the inner surface 201 comprises a transparent lens or cover as discussed generally above. The illumination strip may comprise one or more illumination elements, for example LEDs, positioned along the illumination strip and pointed outward from inner surface 201, towards sleeve 104. In some embodiments, the illuminator unit 100A and/or sleeve 104 may comprise additional lighting elements, light pipes, or reflective materials configured to illuminate the lingual surfaces of one or more teeth.

Sleeve 104 is shown in FIG. 2 comprising facial surface 203, lingual surface 204, and central surface 205. Together, surfaces 203-205 define two channels configured to accept the upper and lower teeth of a subject. The sleeve and the channels may be substantially U-shaped as shown, or may in some embodiments be semicircular or elliptical. In some embodiments, sleeve 104 may have reflective material to disperse light to the lingual, buccal or occlusal teeth surfaces as well as the interproximal spaces between teeth.

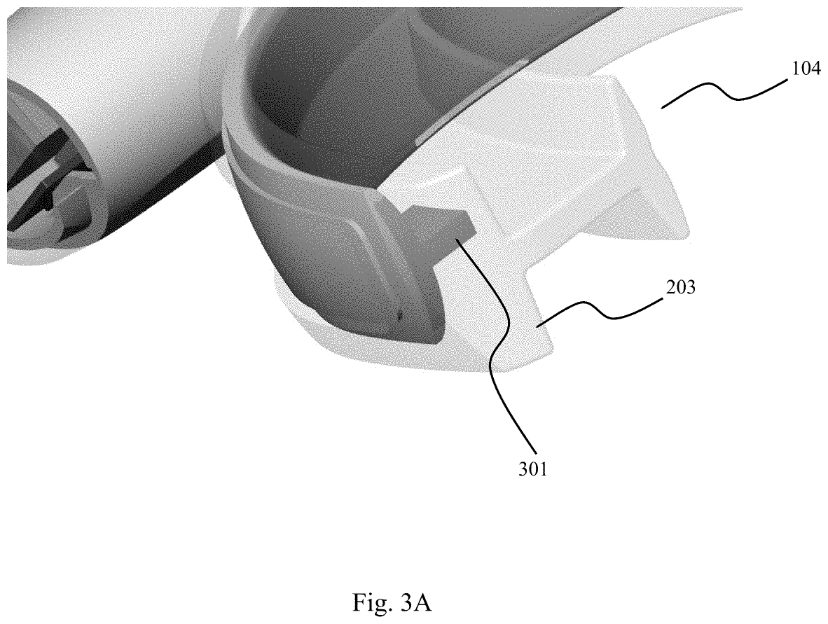

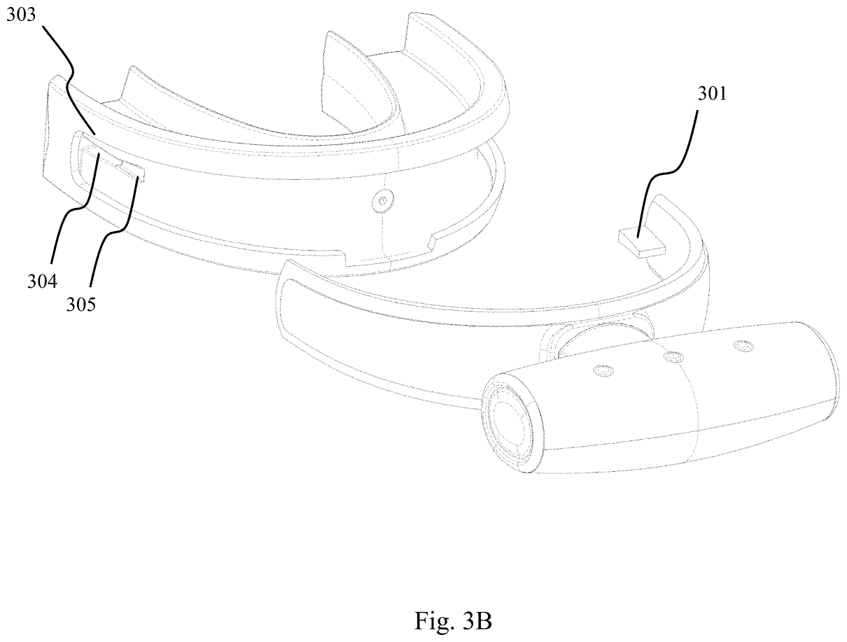

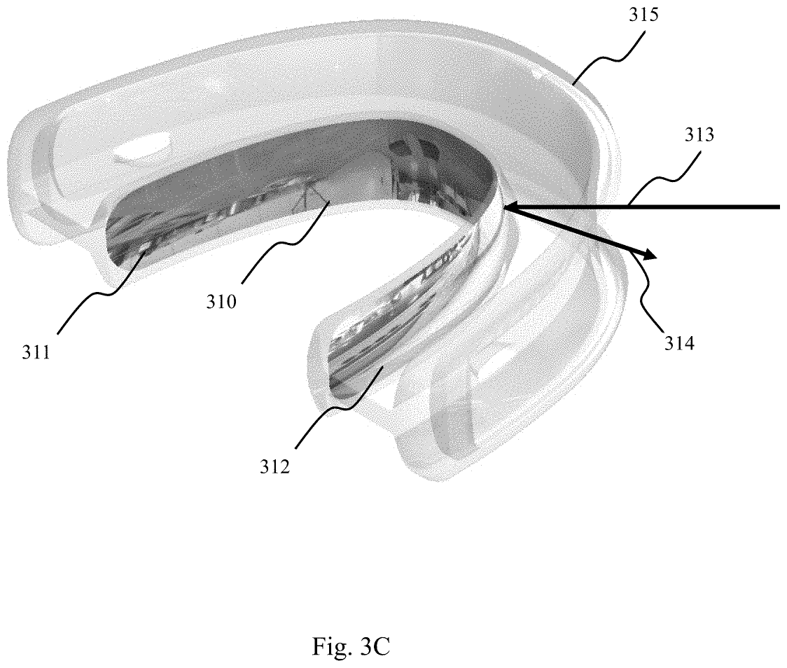

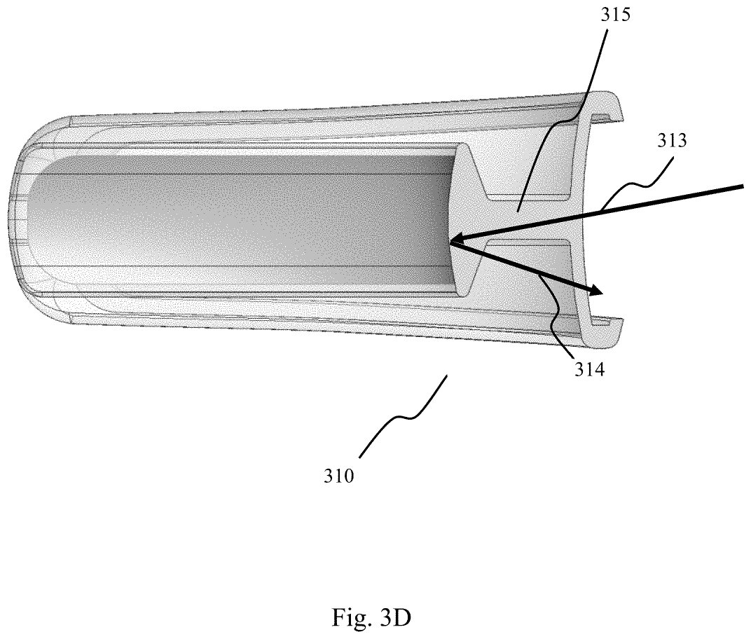

With reference now to FIG. 3A, a detail view of a device of the present invention is shown, focusing on an exemplary fastening element for connecting an illuminator unit and a sleeve. The depicted illuminator unit includes a tab 301 which is configured to mate with a slot (not shown) in the facial surface 203 of sleeve 104. The tab 301 may be rigid or flexible, and may be more or less or equally flexible as the sleeve 104. In some embodiments the tab 301 is made from the same material as the transparent cover or lens of the illumination unit. The tab 301 may be transparent, semi-transparent, or opaque. In some embodiments, a single tab is used on each side of the illumination unit to secure the sleeve 104 in place, but in other embodiments, multiple tabs may be used on each side, for example two or three tabs per side. Tabs may be substantially rectangular as shown in FIG. 3A, but may alternatively be cylindrical, semi-cylindrical, ovular, or semi-ovular. Tabs 301 may have a rounded or beveled edge in order to facilitate insertion into the slots on the sleeve 104.

An alternate view of the tab/slot arrangement is shown in FIG. 3B, where the illuminator unit includes two tabs 301 and the sleeve includes two corresponding slots 303. The slot 303 includes a recessed portion 304 into which the tabs 301 fit when the illuminator unit and sleeve are mated together, as well as a less-recessed guide portion 305 which guides the tab into the recessed portion during mating of the two parts.

Tab 301 and slot 303 may be designed such that the mating surfaces closest to the center of the arch are intentionally manufactured to be in interference with one another. This interference places both the sleeve and the mouth arch under strain when the two are assembled, and leads to an increase in contact pressure between the mating surfaces between the facial surface 203 (see FIG. 2) and the inner illumination strip surface 201 (see FIG. 2). The resultant contact pressure provides a positive seal between the sleeve and the mouth arch.

With reference to FIG. 3C, a variant of a sleeve is shown with a reflective element 310 positioned on lingual surface 204, the reflective element defining a proximal surface 311 and a distal surface 312. In various embodiments, either or both of the proximal surface 311 and the distal surface 312 may be partially or wholly reflective of light in all or a portion of the visible, ultraviolet, and/or infrared spectra. In one embodiment, the distal surface 312 is configured to be reflective, so that light 313 for example supplied by an attached illuminator unit, may reflect along a reflection path 314, for example to provide illumination to the inner (lingual) surfaces of teeth in addition to the facial surfaces. In one embodiment, the reflective element comprises a reflective coating on the lingual wall of the sleeve. In one embodiment, the reflective element may be overmolded into or onto the tray. In one embodiment, the reflective element comprises a reflective material, a woven material (for example a fabric) and/or one or more particles, for example spherical particles.

In some embodiments, the reflective element 310 may be deposited on the sleeve for example via painting, an adhesive, lamination, or heat sealing.

With reference to FIG. 3D, a cross-sectional view of an exemplary sleeve is shown. In some embodiments, in order to maximize light delivery from the buccal/facial side of the teeth, the sleeve may be configured such that the occlusal region 315 of the sleeve 310 is transparent or includes one or more light pipes, fiber optics, chopped optical fibers, or other materials to improve translucency through that part of the tray. In some embodiments, the occlusal region may comprise for example a clear material bite plate that is overmolded, an optically clear silicone, or the like. In some embodiments, the occlusal region may comprise a bite plate that is translucent or configured to diffuse light. In some embodiments, the sleeve 310 includes one or more light pipes configured to direct the light path back toward the lingual surface of the teeth without a separate reflective element.

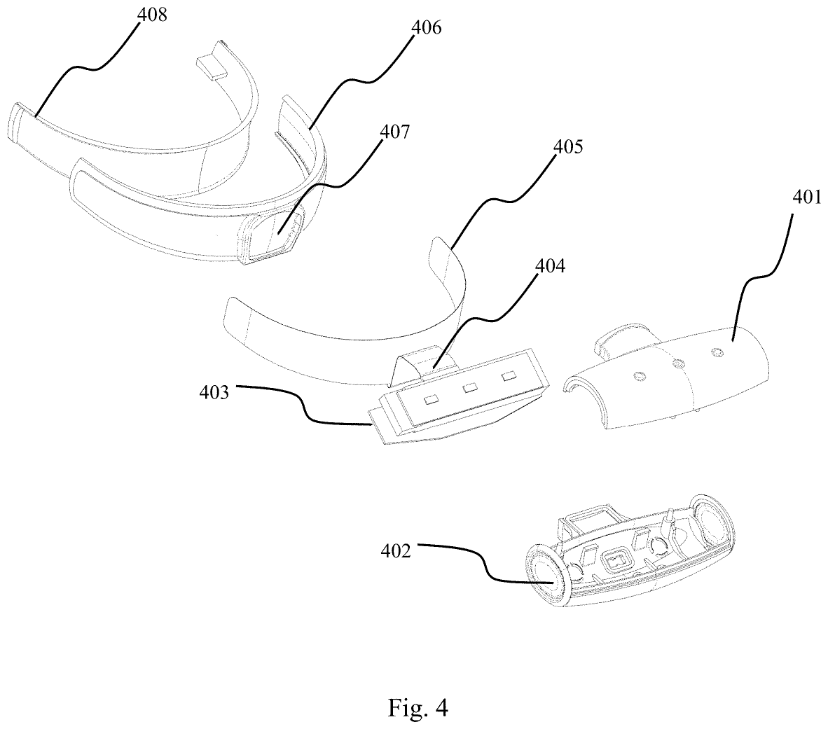

An exploded view of an exemplary device of the present invention is shown in FIG. 4. The depicted view shows a housing for the handle having two halves, an upper half 401 and a lower half 402 which may connect together via snapping, one or more fasteners, an adhesive, or any other suitable means of connection. The housing 401-402 surrounds electronics 403, which include all suitable electronics and control systems for the device, including but not limited to an energy storage unit, a controller unit, support circuitry for illuminating elements, charging circuitry, indicator LEDs, human interface inputs, data logging, data transmission, etc.

The electronics 403 are connected to an illumination element 405 via a flexible circuit board or wire bus 404. Flexible wire bus 404 may provide power and control signals to one or more illuminating elements located in illumination element 405. In some embodiments, illumination element 405 is a flexible circuit board including one or more LEDs mounted thereon.

The illumination unit housing includes a tooth-facing half 408 and a lip-facing half 406, with the lip-facing half including a channel 407 through which the flexible wire bus 404 passes. In the depicted embodiment of FIG. 4, the illumination element 405 is mounted between the tooth facing half 408 and the lip-facing half 406 of the illumination unit housing, with the one or more LEDs pointing toward the tooth-facing half.

In some embodiments, a device of the present invention includes an ambient light sensor, for example comprising a photodiode, mounted on a surface of the device that would typically be located inside the mouth of a subject while in use. The controller could then periodically poll the ambient light measured by the ambient light sensor, and turn off any LEDs or illuminating elements in the illumination unit when the ambient light sensor measures a level of ambient light above a pre-determined threshold (thereby indicating that the illumination unit is not positioned in the mouth of the subject).

Suitable energy storage units include batteries, for example rechargeable or single-use replaceable batteries. In one embodiment, the energy storage unit is a rechargeable lithium-polymer battery. In one embodiment, the battery is a lithium ion battery. In some embodiments, disposable alkaline batteries may be used. Suitable batteries may have a capacity of at least 100 mAh, at least 150 mAh, at least 200 mAh, or at least 250 mAh. In some embodiments, a battery suitable for use with the device supports a high discharge rate, for example 10 C, 15 C, or 20 C. In some embodiments, an energy storage unit may include one or more capacitors or supercapacitors, either in addition to or instead of batteries. In one embodiment, the device may include a charging system for a rechargeable battery, capacitor, or supercapacitor, including for example an outlet to receive a battery charging cable. In some embodiments, a device of the present invention includes an inductive or wireless charging system, configured to charge the energy storage unit when an energy receiving coil located in the device is in proximity to an energy transmitting coil in a separate wireless charging dock or pad.

Suitable controllers include embedded micro-controllers or systems-on-a-chip (SoCs). Controllers may include a quantity of non-transitory computer readable memory with instructions stored thereon, the instructions comprising one or more processes to be executed by a processor to perform computer-enabled methods of the invention. Controllers may include one or more wireless or wired communication interfaces, for example a Bluetooth, WiFi, or other wireless communication interface, allowing the device to be paired to a second external computing device, for example a smartphone. When a device having a controller is configured to communicate with a second computing device, some or all of the computing operations performed by the controller may be distributed to the second computing device to be processed or calculated thereon. In some embodiments, a controller may be configured to connect wirelessly to a remote computing device in order to transfer usage and/or other data to the remote computing device.

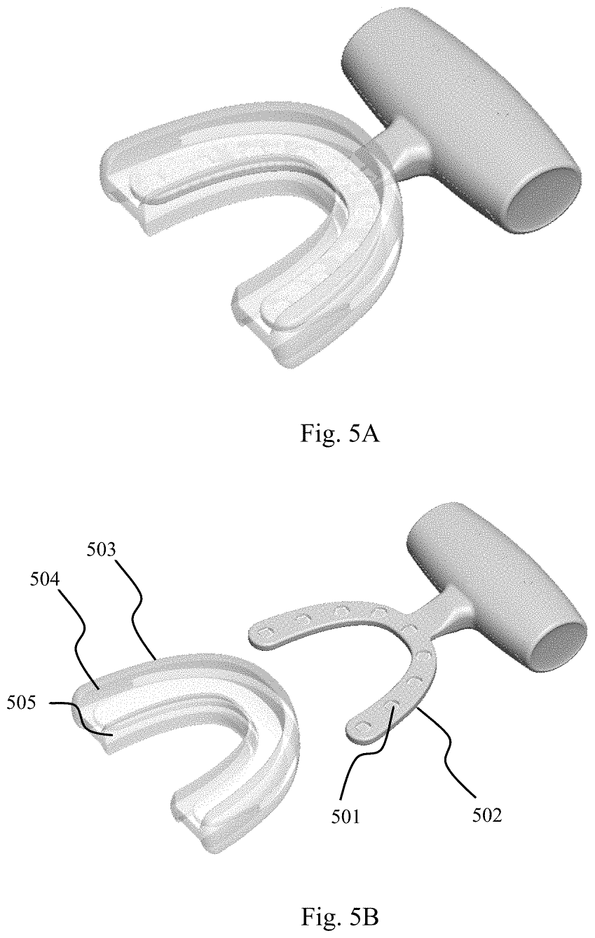

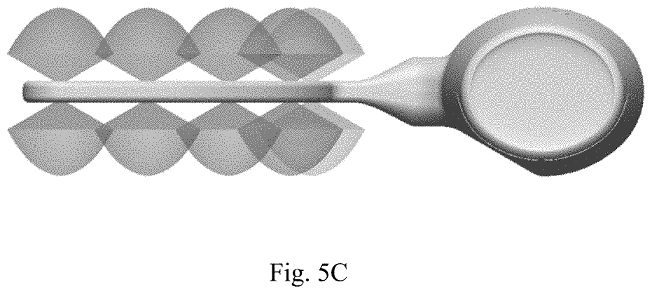

With reference to FIG. 5A and FIG. 5B, another embodiment of the device includes illumination elements 501 positioned on the occlusal plane of a bite tray 502 removably positioned inside a sleeve 503. In the depicted device, light delivery may be varied by physical placement of the one or more lighting elements on the occlusal plane, adjusting the viewing angle of the light sources, for example using interchangeable lenses, or using different sleeve materials or geometry to scatter, focus, or redirect the light. Illumination elements 501 may further comprise a transparent cover or lens positioned over the illumination elements, between the one or more LED elements and the sleeve 503. In some embodiments, the transparent cover or lens is placed in direct contact with the sleeve 503 when the sleeve 503 is connected to illumination elements 501. The cover may be overmolded or may be a separate piece removably or fixedly attached to the illumination elements 501. In some embodiments, the transparent cover or lens may include features or textures configured to distribute or diffuse light from the illumination elements. In some embodiments, one or more surfaces of the sleeve 503 may comprise a reflective material, for example the proximal (inner) surface of the facial portion 504 and/or the distal (outer) surface of the lingual portion 505, in order to direct more light toward the surfaces of the teeth.

A cross-sectional view of the embodiment of FIG. 5A and FIG. 5B is shown in FIG. 5C, illustrating the viewing angles of the illumination elements emanating from the bite plane.

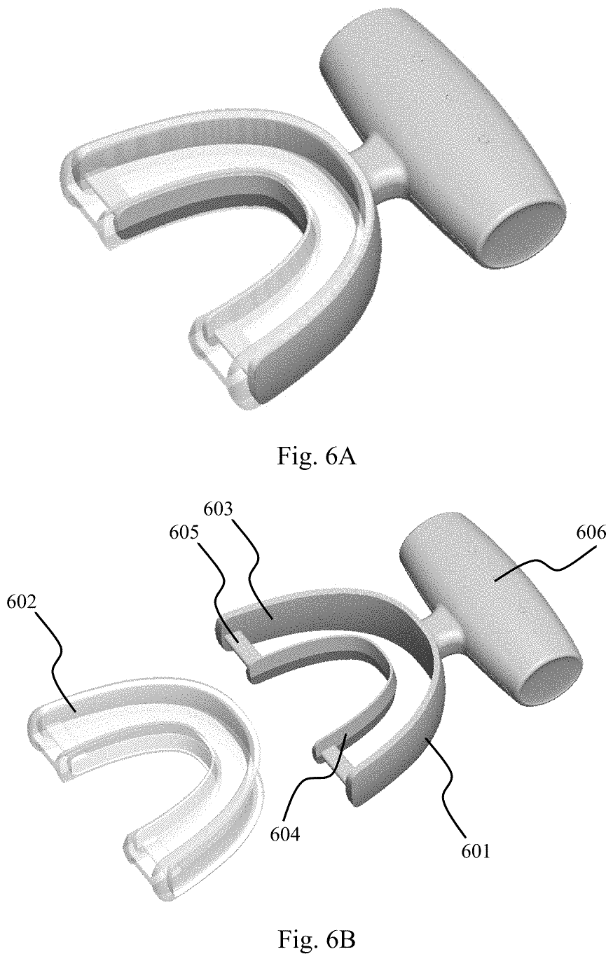

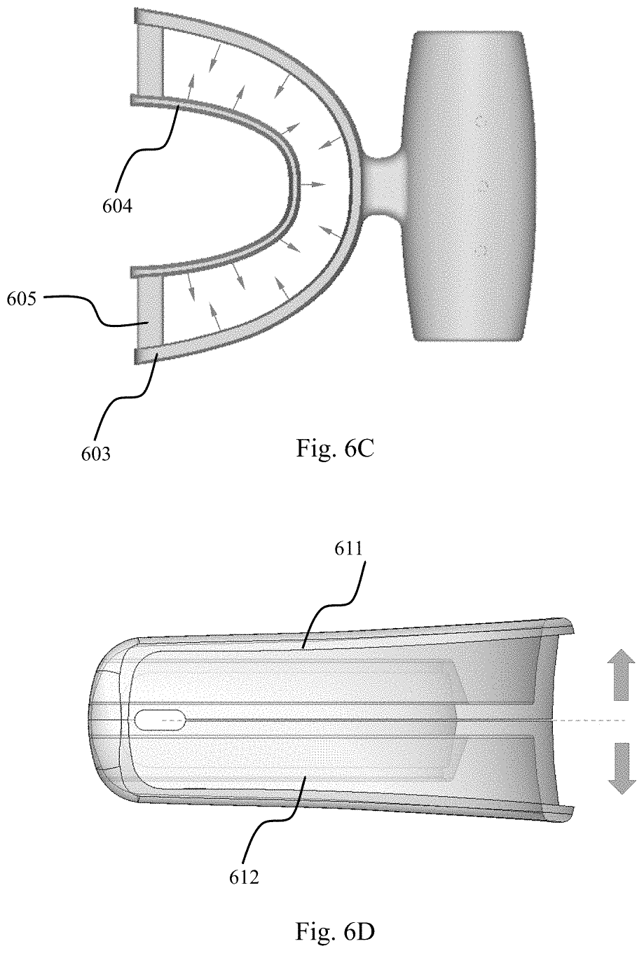

Another embodiment of a device is shown in FIG. 6A, FIG. 6B, FIG. 6C, and FIG. 6D. The depicted device includes an illumination device 601 having a facial portion 603 and a lingual portion 604, with active illumination elements positioned on both. A first set of illumination elements is positioned on the proximal (inner) surface of the facial portion, configured to illuminate at least the facial surfaces of the teeth of a subject. A second set of illumination elements is positioned on the distal (outer) surface of the lingual portion, configured to illuminate at least the lingual surfaces of the teeth of a subject. The facial and lingual portions are joined by two bridging portions 605, which include one or more conductive elements embedded within (not shown) to deliver power and/or control signals to the illumination elements on the lingual portion from the power source which may in some embodiments be positioned in the handle 606.

An overhead view of the device of FIG. 6A and FIG. 6B is shown in FIG. 6C. The arrows depict exemplary illumination paths from illumination elements positioned in the facial 603 and lingual 604 portions of the device.

In one embodiment, a removable/replaceable sleeve (FIG. 6D) for use with the device of FIG. 6A-FIG. 6C may be configured as a clamshell, with upper 611 and lower 612 portions, which may in some embodiments be connected via a hinge, or may alternatively be configured as two separate pieces which snap together to envelop the portion of the illumination device positioned within the mouth of the user, as shown in FIG. 6A. In some embodiments, the sleeve in FIG. 6D includes one or more reflective elements for further guiding the illumination of the teeth, as discussed elsewhere herein.

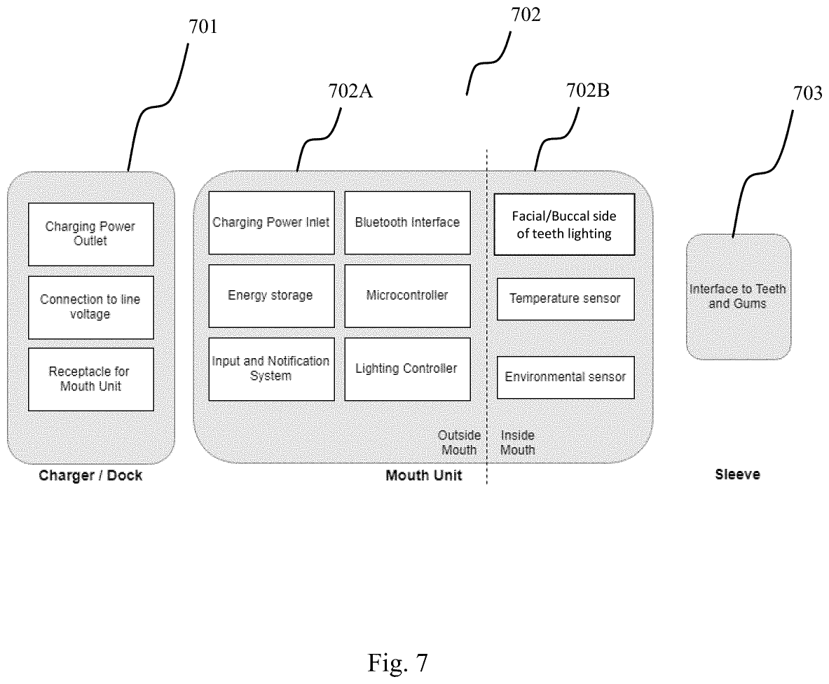

With reference now to FIG. 7, a system diagram of an exemplary device of the present invention is shown. The depicted system diagram is divided into charger/dock 701, mouth unit 702, and sleeve 703. Note that the device 100A of FIG. 1B is an example of mouth unit 702, and sleeve 104 of FIG. 1B is an example of sleeve 703.

In the depicted system diagram, the charger/dock 701 may include a charging power outlet, an electrical connection to line voltage, and a receptacle for the mouth unit. The electrical connection to line voltage may be a connection to an AC power source, for example a mains electrical connection, or may alternatively be a connection to a DC power source, for example a USB connection. Where AC power is used as the power source, an AC/DC converter may either be incorporated into the charger/dock, or may be located separate from the charger/dock, for example with a wall AC plug adaptor. The charging power outlet may be a physical connection point without a wired electrical connection, for example for a wireless charging system, or may alternatively comprise an electrical connection, for example bare contacts, spring contacts, or an electrical connector (for example a barrel power connector). Suitable wireless charging systems include, but are not limited to charging coils or galvanic connections with a magnetic attachment. In some embodiments, a charging coil may have a coil diameter of less than 30 mm, less than 20 mm, or 15 mm or less. In some embodiments, a wireless charging element may be capable of transmitting power at a rate of 250 mW, 500 mW, 1 W, or 2 W. In some embodiments, the mouth unit 702 and charging dock 701 may be configured such that it is physically impossible for the mouth unit 702 to be electrically connected to the charging dock 701 while the mouth unit is positioned in the mouth of a subject.

The charger/dock may also include a receptacle for the mouth unit, for example to hold the mouth unit in place for optimal wireless charging. The receptacle provides a way to store the mouth unit in a way that allows for charging, communicating notifications to the user and minimizing the risks of contamination of the mouth unit. The charger/dock may further comprise a drip catch or small cavity positioned such that any fluid (water, saliva, etc.) dripping from the mouth unit 702 will flow into the drip catch.

The mouth unit 702 is subdivided into two sub-units, with sub-unit 702A located outside the mouth and sub-unit 702B located inside the mouth. Sub-units 702A and 702B may be physically and electrically connected to one another, with one part of the mouth unit protruding from the mouth of the subject while in use. The sub-unit 702A comprises a charging power inlet which is configured to receive power, via a wired and/or wireless connection, from the charging power outlet in charger/dock 701. The charging power inlet is configured to accept power without exposing live conductors that could discharge current in an unplanned manner. The charging power inlet may also be designed to be substantially smooth, so as to prevent microbe, bacteria and/or fungus growth.

An energy storage unit may be included in the mouth unit as discussed above. An input and notification system may comprise any or all control or indicator elements positioned on the mouth unit, including but not limited to buttons, LED indicators, switches, etc. as discussed above.

The depicted mouth unit may include a Bluetooth, WiFi, and/or other wireless communication interface for communication with external computing devices. In some embodiments, a Bluetooth interface in the mouth unit may additionally include a Bluetooth host interface for connecting auxiliary external sensors or indicators. In some embodiments, the Bluetooth interface enables connectivity with a smartphone app, for example to collect and send data from the mouth unit to a smartphone app. The Bluetooth interface may include a separate stand-alone interface, or may be integrated into a system-on-a-chip with a microcontroller. The Bluetooth interface may have a separate physical antenna or may alternatively have an antenna integrated into the same package as the transceiver. The microcontroller may be any suitable microcontroller as discussed above, and the lighting controller is electrically connected to the facial or buccal lighting elements in 702B, which provide illumination to the teeth and gums. The lighting controller may include a fault-detection system for detecting and reporting a failure of the lighting system. Exemplary fault-detection systems include undercurrent/overcurrent monitoring, undervoltage/overvoltage monitoring, or ambient light sensing.

The inside mouth sub-unit 702B includes facial or buccal lighting elements, which may be LEDs, OLEDs, or any other suitable illumination elements as discussed above. The inside mouth sub-unit 702 may additionally include one or more sensors, for example a temperature sensor, thermal sensor, infrared sensor, resistive sensor, environmental sensor, an ambient light sensor, accelerometer, or sensors configured to monitor plaque build-up, teeth discoloration, enamel health, gingival health, or movement of teeth in brace wearers. Lighting elements for use with the present invention may be selected and configured to deliver a minimum light intensity to the faces of the subject's teeth, for example at least 30 mW/cm², at least 35 mW/cm², at least 40 mW/cm², at least 45 mW/cm², at least 48 mW/cm², at least 50 mW/cm², at least 60 mW/cm², or at least 70 mW/cm². Lighting elements may be positioned such that they are at a distance of 1.5 cm or less from the subject's teeth. Where multiple discrete lighting elements are used, the lighting elements may be positioned at a spacing of 3 cm or less from one another, or 2 cm or less from one another. In some embodiments, one or more lighting elements may be positioned immediately adjacent to one another.

Facial lighting elements may be configured to deliver light within a pre-determined wavelength range, for example 200-740 nm, 400-500 nm, 425-475 nm, or 445-465 nm. In some embodiments, facial lighting elements may be configured to deliver light across the entire electromagnetic spectrum, the entire visible light spectrum, the infrared and/or ultraviolet spectra, or any combination of these. In some embodiments, some or all lighting elements in any embodiment described herein may be configured to deliver sustained illumination, pulsed illumination, or a combination thereof. Although energy delivery elements may be referred to herein as “illumination elements,” it is understood that embodiments of the disclosed devices may be configured to deliver electromagnetic energy from a wide range of frequencies along the electromagnetic spectrum, including but not limited to X rays, gamma rays, microwaves, radio waves, or a combination of these.

A temperature sensor may be positioned on the inside mouth sub-unit as a safety precaution to ensure no discomfort to the user. The temperature sensor may be configured for example to detect if the lighting system generates too much heat for the user. The controller may be configured to measure the values from the temperature sensor and interrupt operation of the lighting system if the temperature sensor measures a temperature that is above a predetermined threshold for a predetermined time interval. Suitable temperature thresholds include, but are not limited to, 40° C., 45° C., or 50° C. Suitable time intervals include, but are not limited to, one second, 5 seconds, 10 seconds, 30 seconds, one minute, or any time interval in between. Alternatively, a temperature sensor may be included in circuitry configured to interrupt power to the lighting system independently of the controller, for example as an input to a relay or transistor.

One or more environmental sensors may be positioned in the mouth of the subject and configured to only allow the mouth unit 702B to run the lighting system at full intensity when the mouth unit is placed in conditions that resemble the mouth environment. Suitable environmental sensors include ambient light sensors, proximity sensors, humidity sensors, pH sensors, resistive sensors, etc. In some embodiments, ambient light sensors may be configured to measure light in a frequency band that is isolated as possible from the frequency of light emitted by the facial lighting system. The ambient light sensor may be configured either by a photodiode material selection (i.e. selection of a light sensor with a narrow band detector) or by using a wide band detector with a light filter configured only to permit light in the desired frequency range to reach the light sensor.

The sleeve 703 provides a replaceable physical interface between the electronics of the inside mouth sub-unit 702B and the teeth and/or gums of the subject. The interface in contact with the teeth and soft parts of the mouth may comprise a flexible biocompatible sleeve. The purpose of the sleeve is to provide a comfortable cover to the mouth unit that can be replaced if needed (e.g. it is soiled, discolored or damaged, etc.). The silicone should be transparent or translucent to the light emitted by the lighting system.

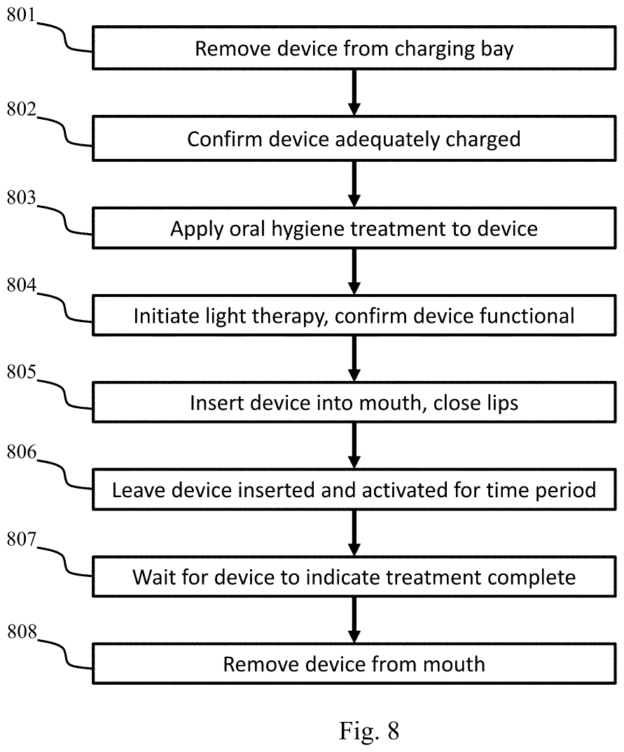

The present invention also includes one or more treatment methods or methods of using the devices and systems described herein. One treatment method, shown in FIG. 8, comprises the following steps, and may be performed before or after a subject completes their typical oral health routine (e.g. brushing/flossing/rinsing). The method of FIG. 8 comprises the steps of removing the device from the charging bay in step 801, confirming the device is adequately charged by inspecting indicators in step 802, applying an oral hygiene treatment to the top and bottom top of the device in step 803, initiating the light therapy and confirming that the device is functional in step 804, inserting the device into the subject's mouth in step 805, leaving the device in the subject's mouth for a predetermined time period in step 806, waiting for an indication that the treatment is complete in step 807, and removing the device from the subject's mouth in step 808.

In some embodiments, a method includes removing, cleaning, replacing, and/or applying a disposable or re-usable sleeve to a mouth unit as described above. In some embodiments, a method of using a device of the invention may include the steps of creating or obtaining a custom-sized sleeve manufactured to fit the mouth of a specific subject, which is configured to physically connect to the mouth unit to ensure a proper fit. In some embodiments, a method of using a device of the invention may include the step of obtaining a sleeve fitted to the subject from a set of standard sized sleeves.

In another embodiment, a method of the present invention may include the steps of waiting for a signal indicating that a device has been removed from a charger, indicating to a user when the device is ready to begin treatment, accepting a user input indicating the beginning of treatment, supplying electrical power to one or more illuminating elements at a first power level, waiting for one or more signals which, individually or in combination, indicate that the device is positioned in the user's mouth, supplying electrical power to one or more illuminating elements at a second power level, waiting for a predetermined time period, turning off the one or more illuminating elements, and indicating to the user via an indicating means that the treatment is complete.

In some embodiments, the treatment duration may be 10 seconds, 20 seconds, 30 seconds, 45 seconds, 60 seconds, 120 seconds, or any suitable duration depending on the subject. In some embodiments an energy storage unit may have a limited capacity capable of treatment for a maximum of 5 minutes, 10 minutes, 20 minutes, or less on a single charge.

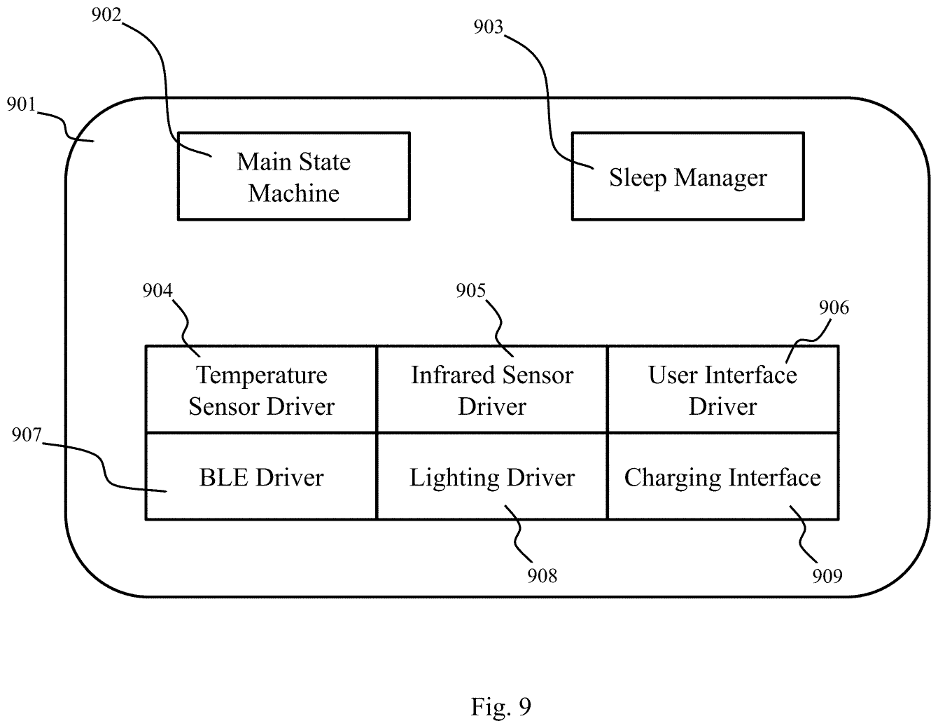

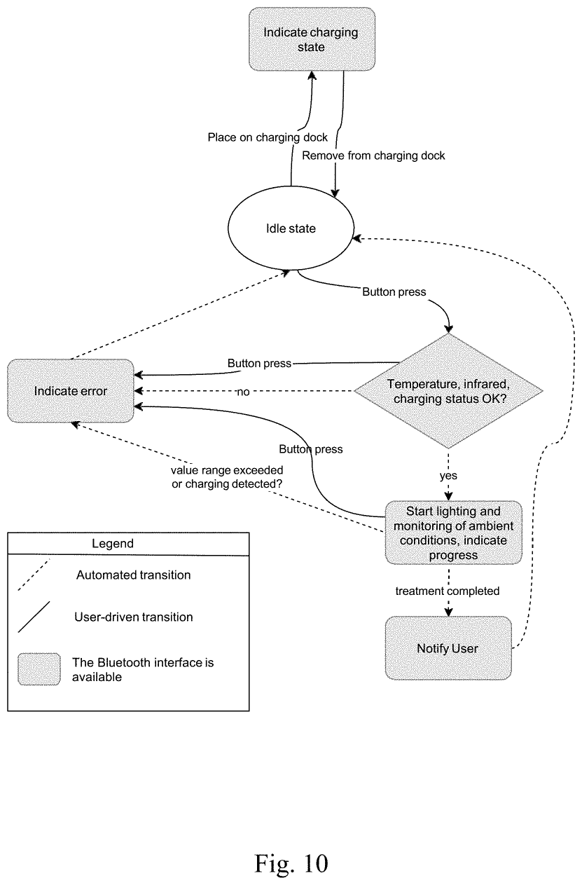

Various methods of the present invention may be implemented in whole or in part as software executed on a computing device. An exemplary software system diagram is shown in FIG. 9. The depicted software 901 includes a series of interconnected modules, including but not limited to a main state machine 902, a sleep manager 903, a temperature sensor driver 904, an infrared sensor driver 905, a user interface driver 906, a BLE driver 907, a lighting driver 908, and a charging interface 909.

The main state machine 902 is the primary module that oversees the device's various functionalities. It is responsible for operating the user interface, reading the temperature and the ambient infrared light levels, managing the BLE and the RF charging interface, controlling the lighting system, and keeping a low power consumption. FIG. 10 shows a state diagram of an exemplary main state machine.

The temperature sensor driver 904 communicates with the temperature sensor. The temperature sensor is used to make sure that the mouth unit has a temperature compatible with being in the mouth prior to starting a treatment. During the treatment, the temperature sensor is used to trigger an alert if a threshold temperature is exceeded. Once an alert occurs, the lighting is turned off to avoid further heating.

The infrared sensor driver 905 works in conjunction with the lighting driver 908 to make sure that the measured infrared light intensity prior to starting a treatment are compatible with the mouth unit being located in the mouth (i.e. very low). During a treatment, the infrared sensor driver will periodically turn off the lighting, monitor the infrared light intensity, and turn the lighting back on such that the lighting system does not interfere with the infrared readings, and the duty cycle of the lighting system is as high as possible.

The user interface driver 906 reads button or other inputs and lights the signaling LEDs or other indicators according to the different states of the main state machine 902. This module also can control a vibration motor to further notify the user. The BLE driver 907 provides the custom BLE service with GATT attributes matching the reporting requirements of a corresponding smart phone application. The lighting driver 908 turns on and off the LEDs in the mouth unit according to the different states of the main state machine. The charging interface 909 monitors the charging interface and reports the charging state to the main state machine 902. Finally, the sleep manager 703 works in collaboration with the main state machine 902 to put the microcontroller to sleep as frequently as possible to conserve power in the energy storage unit while ensuring that the user interface remains responsive.

Some or all steps of a method of the invention may be executed on a separate computing device, for example an app running on a smartphone or other portable computing device, or a web portal interfacing with a server or cloud instance, communicatively connected to the mouth unit via a wireless data connection. An exemplary smartphone app may allow for monitoring functionality, for example displaying battery status, a treatment timer, sensor indicators, a treatment ready indicator, a treatment complete indicator, or a user compliance tracker or indicator. An exemplary smartphone app may also provide for control signals to be sent from the app to the device, for example tapping a button in the app to begin or pause treatment, or initiating an audible or visual location beacon on the device (e.g. “find me” functionality). Additionally, an exemplary smartphone app may provide for reporting and compliance monitoring functionality. For example a smartphone app may collect sensor information or treatment information from a connected treatment device, then transmit some or all of the collected information to a caregiver in order to validate that a treatment regimen provided by the caregiver is being followed. In some embodiments, a smartphone app may provide periodic reminders to a user, a user's guardian, and/or a user's caregiver (for example a parent) to administer a treatment according to a predetermined schedule.

Exemplary smartphone apps or web portals may further connect a device of the invention to the Internet, for example via home WiFi or via Bluetooth, and may be used to create reminders for a user to apply a treatment, for example via smartphone or e-mail notifications or alerts. In some embodiments, an app or portal may be configured to create or display a report card of compliance data for a time period to a user or a guardian, caregiver, or parent of the user. An app or portal may provide compliance data to an orthodontic practice. In response to the compliance data, the orthodontic practice, its systems or agents may notify or send reminders to the user or caregiver/guardian, for example to use the device, or to remind the user of the consequences of not using the device. In some embodiments, an app or portal may save compliance data related to one or more users, at least for the duration of one or more orthodontic treatments. In some embodiments, an app or portal may prompt a caregiver or orthodontist to confirm whether or not white spot lesions formed during treatment. In some embodiments, a portal or all may provide inventory control or ecommerce functions, for example allowing a user to order additional supplies, or to check inventory of particular parts.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An oral hygiene device, comprising:
a housing having an outside mouth portion and an inside mouth portion;
at least one energy delivery element disposed in or on the inside mouth portion, configured to deliver energy to one or more teeth of a subject;
a controller disposed in the outside mouth portion electrically connected to the at least one energy delivery element;
an energy storage unit disposed in the outside mouth portion; and
a sleeve removably connected to the inside mouth portion of the housing, the sleeve having upper and lower channels configured to accept the upper and lower teeth of the subject.

2. The oral hygiene device of claim 1, further comprising at least one control element disposed on the outside mouth portion.

3. The oral hygiene device of claim 1, further comprising a wireless communication interface electrically connected to the controller and configured to facilitate communication between the controller and computing device external to the oral hygiene device.

4. The oral hygiene device of claim 1, further comprising at least one indicator element disposed in the outside mouth portion.

5. The oral hygiene device of claim 4, wherein the indicator element is an LED.

6. The oral hygiene device of claim 1, wherein the at least one energy delivery element is a plurality of LEDs.

7. The oral hygiene device of claim 6, wherein the LEDs are configured to emit light in a range of 200 nm to 1500 nm.

8. The oral hygiene device of claim 7, wherein the LEDs are configured to emit light in a range of 400 nm to 500 nm.

9. The oral hygiene device of claim 6, wherein the LEDs are configured to deliver a light intensity of at least 30 mW/cm² to teeth of the subject.

10. The oral hygiene device of claim 1, further comprising a temperature sensor disposed in the inside mouth portion and communicatively connected to the controller, wherein the controller is configured to interrupt a treatment if a temperature measured by the temperature sensor exceeds a threshold.

11. The oral hygiene device of claim 1, further comprising at least one environmental sensor disposed in the inside mouth portion and communicatively connected to the controller, wherein the controller is configured to start a treatment if at least one measurement obtained from the at least one environmental sensor indicates that the oral hygiene device is positioned in a mouth of the subject.

12. The oral hygiene device of claim 11, wherein the at least one environmental sensor comprises a light sensor.

13. The oral hygiene device of claim 1, wherein the energy storage unit is a rechargeable battery.

14. The oral hygiene device of claim 13, further comprising a wireless charging system configured to charge the battery.

15. The oral hygiene device of claim 1, wherein the sleeve includes at least one reflective element.

16. The oral hygiene device of claim 1, wherein the at least one energy delivery element is positioned in the occlusal plane of the teeth of the subject.

17. The oral hygiene device of claim 1, wherein the at least one energy delivery element comprises a first energy delivery element configured to deliver energy to a facial surface of the teeth of the subject; and
a second energy delivery element configured to deliver energy to a lingual surface of the teeth of the subject.

18. The oral hygiene device of claim 1, wherein the at least one energy delivery element is configured to deliver pulsed energy to the one or more teeth of the subject.

19. The oral hygiene device of claim 1, wherein the energy delivery element is configured to deliver electromagnetic energy in a spectrum selected from the group consisting of gamma rays, x-rays, ultraviolet light, visible light, infrared light, microwaves, and radio waves.

20. A method of using an oral hygiene device, comprising:
applying a light activated treatment material to an oral hygiene device;
waiting for an indication that the oral hygiene device is ready to begin treatment;
activating a control element on the oral hygiene device to start an oral hygiene treatment;
inserting the oral hygiene device into a mouth of a subject;
waiting for an indication that the treatment is complete; and
removing the oral hygiene device from the mouth.

21. The method of claim 20, further comprising the step of applying a sleeve to the oral hygiene device.

22. The method of claim 21, further comprising the step of obtaining a sleeve custom manufactured for the subject or fitted to the subject from a set of standard sized sleeves.

23. The method of claim 20, further comprising the step of charging an energy storage unit in the oral hygiene device.

24. A method of administering an oral hygiene treatment, comprising:
waiting for a signal indicating that a device has been removed from a charger;
indicating to a subject when the device is ready to begin treatment;
accepting a user input from the subject indicating the beginning of treatment;
supplying electrical power to one or more energy delivery elements at a first power level;
waiting for one or more signals which, individually or in combination, indicate that the device is positioned in the subject's mouth;
supplying electrical power to the one or more energy delivery elements at a second power level;
waiting for a predetermined time period, turning off the one or more energy delivery elements; and
indicating to the subject via an indicating means that the treatment is complete.

25. The method of claim 24, wherein the predetermined time period is two minutes or less.

26. The method of claim 24, further comprising:
monitoring a temperature measured by a temperature sensor positioned on the device; and
turning off the one or more energy delivery elements when the temperature exceeds a threshold.
